(19) United States
(12) Patent Application Publication
(10) Pub. No.: US 2006/0029671 A1
(43) Pub. Date: Feb. 9, 2006

(54) Tablet coating composition

(75) Inventors: Susan O. Freers, Muscatine, IA (US); Roger E. McPherson, Muscatine, IA (US)

Correspondence Address: BANNER & WITCOFF, LTD., TEN SOUTH WACKER DRIVE, SUITE 3000, CHICAGO, IL, 60606, US

(73) Assignee: Grain Processing Corporation, Muscatine, IA (US)

(21) Appl. No.: 11/196,479

(22) Filed: Aug. 4, 2005

Related U.S. Application Data
(60) Provisional application No. 60/599,418, filed on Aug. 6, 2004.

(51) Int. Cl. A61K 9/28; A61K 9/24

(52) U.S. Cl. 424/472; 427/2.14

(57) ABSTRACT

Disclosed is a method for coating a tablet with a coating composition. The tablet generally comprises a biologically active material, and generally one or more biologically acceptable excipients. The coating composition comprises hemicellulose, partially depolymerized hemicellulose, or a mixture thereof. In preferred embodiments, the coating composition includes a plasticizer, and a coating composition further may include coloring agents, opacifiers, and other ingredients. Also disclosed is a coated tablet, a method preparing a coating composition, a coating composition, and a coating composition precursor.

This application claims priority to provisional application Ser. No. 60/599,418 filed Aug. 6, 2004, which is hereby incorporated by reference in its entirety.

### TECHNICAL FIELD OF THE INVENTION

The invention is in the field of tablet coating.

### BACKGROUND OF THE INVENTION

In the manufacture of pharmaceutical and other ingestible tablets, the tablets generally are coated with a film-forming polymer before packaging. Uncoated tablets may be difficult to swallow, and the tablet coating is thought to facilitate oral ingesting. The coating also may provide protection from environmental factors, thus improving the stability and the shelf life of the tablets. In addition, many tablets have a unique appearance that includes a specific color coating that enables the consumer to recognize the active ingredient in the tablet. The tablet coating preferably is continuous over the entire tablet.

Coatings typically are applied by spraying a tablet coating composition onto the uncoated tablets and allowing the coating composition to dry. Among the criteria for a tablet coating composition is that the coated tablets must dry satisfactorily and must not agglomerate or exhibit picking or chipping. Where a logo is used, the tablet coating should enable good logo definition, and, if the logo is indented into the tablet, the coating should not fill the indentation. The tablet coating should not come off the tablet during ordinary handling.

Commercially, hydroxypropyl methylcellulose (HPMC) or hydroxypropyl cellulose (HPC) are used as the film-forming polymers in table coating compositions. The prior art has suggested other materials. For instance, gellan gum is suggested in U.S. Pat. Nos. 6,485,747 B1 and 6,395,298. Another document, U.S. Pat. No. 6,326,028, discusses a combination of gellan gum and alginate. Karaya gum, locust bean gum, xanthan gum, gum tragacanth, and sodium alginate are suggested in U.S. Pat. No. 6,309,668. Maltodextrins, which are starch hydrolyzates, are disclosed in U.S. Pat. Nos. 4,828,841; 4,725,441; and 4,643,894.

The present invention seeks to provide a tablet coating composition that is at least as satisfactory as the coating compositions that are commercially available.

### THE INVENTION

It has now been found that hemicellulose, partially depolymerized hemicellulose, and mixtures thereof have excellent properties for use in tablet coating compositions. In accordance with the preferred embodiments of the invention, a method for coating tablets is provided. The tablets are coated with a coating composition that includes hemicellulose, partially depolymerized hemicellulose or a mixture thereof. In preferred embodiments of the invention, the coating composition includes other ingredients, such as a plasticizer. In those embodiments of the invention in which a colored coating is desired, the composition generally includes a coloring agent and an opacifier. The invention contemplates coating only a portion of the tablet with the composition, but in most embodiments the entire tablet will be coated with the composition. The tablet generally comprises a biologically active material, but may be a placebo.

Also encompassed by the invention are a method for preparing a coating composition, a coating composition, a coating composition precursor, and tablets that are coated with a coated composition.

### DESCRIPTION OF THE PREFERRED EMBODIMENTS

The invention contemplates a coating composition that includes hemicellulose, partially depolymerized hemicellulose, and mixtures thereof. Hemicellulose may be obtained from a variety of sources, such as corn hulls, cottonseed hulls, peanut hulls, oat hulls, soybean hulls, palm hulls, coconut hulls, and lees from rice, wheat, beets or potatoes. A preferred hemicellulose is corn hull hemicellulose, which is obtained by treatment of corn hulls. The remaining discussion focuses on corn hull hemicellulose, but it should be understood that hemicellulose obtained from other sources may be used in conjunction with the invention.

The domestic U.S. hybrid corn crop is enormous and stable, and the composition of the corn seeds does not vary significantly. Corn crops provide a reliable, low cost, and consistent source of hulls, bran, and spent germ as byproducts from the production of starch, corn flour, protein and oil. Corn hulls from the corn wet milling industry are a good, inexpensive, source for hemicellulose. An accepted composition of commercially produced corn hulls or corn bran is as follows:

| | |
|---|---|
| Hemicellulose | 56.38% |
| Cellulose | 18.79% |
| Starch | 8.14% |
| Protein | 7.90% |
| Fat | 1.69% |
| Acetic acid | 3.51% |
| Ferulic acid | 2.67% |
| Diferulic acid | 0.58% |
| Coumaric acid | 0.33% |
| Other | (trace) |

Hemicellulose and cellulose together comprise holocellulose. The polymers that comprise holocellulose are made up of simple sugars, such as D-glucose, D-mannose, D-galactose, d-xylose, l-arabinose, d-glucoronic acid, and other sugars such as L-rhamnose and D-fructose. Cellulose is a glucan polymer of D-glucanopyranose units linked together via β-(1-4)-glucosidic bonds. The average DP (degree of polymerization) for plant cellulose ranges from a low of about 50 to about 600. Cellulose molecules are randomly oriented and have a tendency to form inter-and intra-molecular hydrogen bonds. Most isolated plant cellulose is highly crystalline and may contain as much as 80% crystalline regions. The hemicellulose fraction of plants is composed of a collection of polysaccharide polymers with a typical lower DP than the cellulose in the plant. Hemicellulose contains mostly D-xylopyranose, D-glucopyranose, D-galactopyranose, L-arabinofuranose, D-mannopyranose, and D-glucopyranosyluronic acid, with minor amounts of other sugars. The various forms of hemicellulose and the ratio of hemicellulose to cellulose is not well defined and may vary from plant to plant or from crop to crop within a given plant.

Hemicellulose or a hemicellulose-containing material may be obtained from the hulls in any suitable manner. The isolation of corn hull hemicellulose from corn hulls is taught in the technical literature and is taught in the following patents: U.S. Pat. No. 2,801,955, U.S. Pat. No. 3,716,526, U.S. Pat. No. 2,868,778, and U.S. Pat. No. 4,038,481. The treatment of corn hull hemicellulose with xylanase to generate corn hull hemicellulose hydrolyzate is taught in U.S. Pat. Nos. 6,488,754 B2 and 6,179,905.

Generally, the foregoing techniques yield hemicellulose in an aqueous solution. Any aqueous solution of hemicellulose may be employed in conjunction with the invention, but preferably, the hemicellulose solution is that obtained or derived from the soluble component of the alkali digest of cooked corn hulls. This digest typically will include starch (in an amount of 5 to 25%, but generally at least about 5%); protein, hemicellulose, fatty acid salts; glycerin, acetic acid, ferulic acid, diferulic acid, coumaric acid, and trace amounts of other materials such as phytosytosterols and minerals.

The partially depolymerized hemicellulose can be obtained by any suitable method, but preferably is obtained by the partial depolymerization of a soluble hemicellulose precursor. The soluble hemicellulose precursor comprises or is obtained from the hemicellulose-containing soluble phase obtained by hydrolysis of a hemicellulose-containing plant source. In accordance with a highly preferred embodiment of the invention, the partially depolymerized hemicellulose is obtained by the partial depolymerization of a soluble hemicellulose precursor that is substantially completely free of cellulose and other insoluble components from the plant source from which the hemicellulose is obtained, as taught in U.S. Pat. No. 6,063,178. As provided in more detail therein, the hemicellulose precursor most preferably is obtained from a soluble phase extracted from hydrolyzed destarched corn hulls produced by the corn wet milling industry.

In accordance with a preferred embodiment of the invention, hemicellulose is removed from the hemicellulose-containing plant source in a soluble phase. Preferably, at least a majority of the hemicellulose component of the plant source, more preferably substantially all of the hemicellulose portion, is separated from insoluble components of the plant source. For example, when the hemicellulose-containing plant source comprises corn hulls, the soluble phase preferably is extracted from the corn hulls. The hemicellulose is extracted by heating an aqueous alkaline slurry of the corn hulls to a temperature of at least about 130° F. (54.5° C.), more preferably at least about 212° F. (100° C.), for a time sufficient to extract a substantial portion of the hemicellulose and other soluble components from the corn hulls. When the corn hull slurry is heated to boiling at atmospheric pressure, it has been found that the slurry should be heated with agitation for a time of at least about 60 minutes, more preferably at least about 80 minutes, and most preferably at least about 120 minutes, to extract the hemicellulose. This time may be substantially shortened if the corn hull slurry is cooked at higher temperatures under pressure. For example, corn hulls may be cooked at 315° F. (157° C.) at 70 psig for a time of about 5 minutes. Generally, any other reaction conditions as may be found to be suitable may be employed in conjunction with the invention.

Insolubles, for example, cellulose, are then physically removed from the reaction mixture, for example, by centrifugation. The soluble phase will contain hemicellulose and other soluble components. For example, it is believed that the soluble phase will contain protein hydrolyzate, salts of fatty acids, glycerin, and salts of natural acids, such as ferulic acid and coumaric acid. It should be understood that although the foregoing represents the preferred method of obtaining the hemicellulose precursor, any hemicellulose obtained via any method may be depolymerized and incorporated into a coating composition in connection with the invention.

After the hemicellulose precursor is obtained, the soluble hemicellulose and other soluble components of the corn hulls then may be concentrated, or water may be removed substantially completely, such as by evaporation or spray-drying, to provide a solid hemicellulose-containing soluble phase. The hemicellulose in the hemicellulose-containing soluble phase can then be depolymerized in any suitable manner as described hereinbelow, and used in accordance with the present invention. Alternatively, the hemicellulose in the hemicellulose solution may be depolymerized prior to concentration and the resulting product optionally concentrated and used. It is further contemplated that the hemicellulose may be partially depolymerized prior to separation of the hemicellulose in a soluble phase from insoluble portions of a hydrolyzed plant source, although such is not presently contemplated to be preferred.

The hemicellulose can be partially depolymerized by any suitable method known in the art or otherwise as may be found to be suitable. The term “partially depolymerized,” as used herein refers generally to the product obtained when hemicellulose is subjected to a depolymerization reaction under conditions such that a partially depolymerized hemicellulose is obtained. Partial depolymerization of cellulose and hemicellulose are known in the art and can be accomplished, for example, enzymatically or chemically. Enzymatic partial depolymerization is described, for example, in U.S. Pat. Nos. 5,200,215 and 5,362,502. Chemical partial depolymerization is described, for example, in R. L. Whistler and W. M. Curbelt, J. Am. Chem. Soc., 77, 6328 (1955). The product of partial depolymerization of the hemicellulose has not been characterized with certainty, but it is presently believed that partial depolymerization by enzymatic methods occurs via random enzymatic cleavage.

Preferably, the partial depolymerization reaction is carried out enzymatically, i.e., under enzymatic catalysis. In a preferred embodiment, the hemicellulose is partially depolymerized with a xylanase enzyme, such as a xylanase that is active under acidic pH. In such case, the pH of the hemicellulose-rich soluble phase of the alkaline hydrolyzate typically is undesirably high and should be adjusted to a pH at which the depolymerizing enzyme is active. When a xylanase that is active under acidic conditions is used, the xylanase is preferably one which is active in the hemicellulose-containing soluble phase below about pH 7, and is most preferably active in the hemicellulose-containing soluble phase at about pH 4.8. In a particularly preferred embodiment, the enzyme utilized in the enzymatic partial depolymerization reaction is GC-140 xylanase, which is available from Genencor International, Rochester, N.Y.

Enzymatic partial depolymerization of hemicellulose may be regulated by controlling the reaction conditions that affect the progress of the depolymerization reaction, for example, the enzyme dosage, temperature, and reaction time. Monitoring of the depolymerization reaction can be accomplished by any suitable method known in the art. For example, the rate or extent of depolymerization can be measured on the basis of viscosity, which typically decreases as the average molecular weight of hemicellulose product decreases during the partial depolymerization reaction. The viscosity (or the rate of change of viscosity over time) can be measured with a viscometer, for example, the rapid viscometer marketed by Foss Food Tech. Corp., Eden Prairie, Minn.. When a rapid viscometer is used to measure viscosity, it is preferably measured at 25° C. after the solution is allowed to equilibrate thermally for about 15 minutes.

Any enzyme dosage (weight of enzyme relative to the overall weight of solution) as may be found to be suitable for depolymerizing the hemicellulose may be used in connection with the invention. For example, in one embodiment xylanase enzyme is used at a dosage ranging from about 0.1 g to about 0.3 g of xylanase per about 5000 g of hemicellulose solution obtained from a plant source. It will be appreciated that the rate and/or the extent of depolymerization achieved at one enzyme dosage can be increased by using a relatively higher enzyme dosage. In this regard, the reaction time required to achieve partial depolymerization is inversely proportional to the enzyme dosage. It will also be appreciated that the enzymatic partial depolymerization reaction can exhibit a “plateau,” during the course of the enzymatic partial depolymerization reaction at which the average molecular weight of the partially depolymerized hemicellulose (as evaluated, for example, by viscosity measurements) does not substantially continue to decrease as the reaction continues. Typically, the plateau is preceded by a relatively rapid initial rate of partial depolymerization. It has been found, for example, that the partial depolymerization of a soluble phase hemicellulose solution having an initial viscosity of 290 cp (measured with a rapid viscometer) exhibited a plateau at a viscosity of about 199 cp when the enzyme dosage was 0.1288 g enzyme per 5000 g of hemicellulose solution (9.4% solids). However, when an enzyme dosage of 0.2542 g enzyme per 5000 g of solution was employed under similar conditions the reaction exhibited a plateau at a solution viscosity of about 153 cp. It will thus be appreciated that a particular enzymatic reaction may reach a plateau at a different average molecular weight depending on the enzyme dosage or on the particular enzyme used. Preferably, the enzymatic partial depolymerization is allowed to proceed until the plateau is reached.

The reaction may proceed at any suitable temperature. For example, when GC-140 xylanase (commercially available from Genencor International, Rochester, N.Y.) is used, the temperature is most preferably about 59° C., and the reaction time is most preferably about 4 hours when the xylanase dosage ranges from about 0.1 g to about 0.3 g of xylanase per about 5000 g of reaction solution. The enzymatic reaction can be terminated by any suitable method known in the art for inactivating an enzyme, for example, by adjusting the pH to a level at which the enzyme is rendered substantially inactive; by raising or lowering the temperature, as may be appropriate, or both. For example, xylanases that are active at acidic pH's can be inactivated by raising the pH to about 7.2 and simultaneously raising the temperature to about 90° C.

In accordance with the invention, the coating composition is used to coat a tablet. The tablet comprises any biologically active material, biologically inert material, or mixtures thereof. Generally, the tablet will include the biologically active material in combination with one or more inert or nearly inert excipients, although it is contemplated that the tablet may comprise solely inert ingredients (i.e., a placebo). The invention is not deemed to be limited in scope to any particular biologically active materials, but to the contrary any suitable material might be used in conjunction with the invention. Examples of such materials include pharmaceutically active ingredients, over-the-counter drugs and medicines, vitamins, nutritional supplements, minerals, and so forth. Examples of drugs used in conjunction with the invention include analgesics, steroids, antihistamines, decongestants, expectorants, and so forth. Prodrugs are deemed to be within the scope of the term “biologically active material.” More generally, any suitable biologically active material may be used in conjunction with the invention. Any suitable excipient may be used in connection with the invention.

A coating composition useful in conjunction with the invention includes water and hemicelluloses, partially depolymerized hemicellulose, or a mixture thereof. Preferably, the hemicellulose or partially depolymerized hemicellulose are present in the composition in an amount ranging from about 10 to 20% by weight with the balance being water and other ingredients. Other amounts of hemicellulose or partially depolymerized hemicellulose may be used if desired. If a mixture of hemicellulose and partially depolymerized hemicellulose is used in conjunction with invention, preferably the total amount of such material is in the range of 10 to 20%. Any suitable ratio of hemicellulose to partially depolymerized hemicellulose may be used in conjunction with the invention.

The depolymerization of the hemicellulose may proceed to any suitable extent. Generally, it is desired that the partially depolymerized hemicellulose will still have a film-forming property. It is desired to partially depolymerize the hemicellulose in conjunction with the invention to achieve a lower viscosity than that of an otherwise similar hemicellulose, as evaluated in an aqueous solution at the same solids content and temperature. Hemicellulose derived from corn often have a molecular weight in the range of 220,000 Daltons; it is believed that partial depolymerization of this material to an average molecular weight of 70,000 Daltons will provide a partially depolymerized hemicellulose that is suitable for use in conjunction with the invention. In some embodiments of the invention, the hemicellulose may be partially depolymerized to a greater or lesser extent.

The coating composition also preferably includes a plasticizer. In accordance with these embodiments of the invention, the plasticizer is any material suitable for rendering film formed by the hemicellulose or partially depolymerized hemicellulose more flexible. Preferred plasticizers include polyethylene glycol (preferably having a molecular weight of 3350), propylene glycol and glycerin. More generally, other suitable plasticizers may be used in conjunction with the invention. The plasticizer is preferably present in an amount of about 10 to 20% by weight of the hemicellulose or partially depolymerized hemicellulose. For instance, if the coating composition includes 10% by weight of partially depolymerized hemicellulose, the plasticizer is preferably present in the amount of about 1 to 2% by weight of the composition. Other suitable plasticizers or other suitable percentages of plasticizers may be used in conjunction with the invention.

The coating composition further may include a detackifier. The detackifier may be present in any amount suitable to reduce tackiness of the dried coating composition relative to an otherwise identical composition prepared in the absence of the detackifiers. It is contemplated that the detackifier may include material such as talc, polysorbate 80, and powdered starches, such as PURE-DENT C815, a powdered starch sold by Grain Processing Corporation of Muscatine, Iowa. The detackifier may be present in any suitable amount; it is contemplated that the amount of the detackifier will vary depending on the percentages of the other ingredients in the coating composition and on the nature of the detackifier. The detackifier is preferably present in an amount of 0.25-2% by weight.

It is contemplated that the coating composition may be a clear composition or a colored composition. When a colored coating composition is desired, the composition preferably includes a coloring agent, which may be any biologically acceptable dye, pigment, lake or the like. The coloring agent may be present in any suitable amount, such as an amount ranging from 0.05 to 2% by weight. It is frequently contemplated that titanium dioxide or another opacifier may be used in conjunction with the invention. The opacifier may be present in any suitable amount, preferably, an amount ranging from about 0.05 to 1% by weight. If a white coating is desired, titanium dioxide is preferably employed as a white pigment.

The coating composition may be provided to have any viscosity suitable for use in conjunction with tablet coating. The preferred viscosity is 310 cp (Brookfield Viscosity, 24° C. at 100 rpm with No. 4 spindle). It is contemplated that the viscosity may be lower or higher than this value, and successful results have been observed at 90 centipoises and as high as about 500 centipoises. It is contemplated that the exact viscosity of a particular coating composition will be selected by one of ordinary skill in the art depending on the particular coating equipment and the ingredients employed in conjunction with the invention.

The coating composition may be applied to the tablet in any suitable amount. Preferably, coating composition is applied in an amount ranging from about 0.5 to 5% by weight of the uncoated tablet. Generally, colored coating compositions are applied in greater amounts than clear compositions. It is preferred that, when a clear composition is employed, the composition should be applied in an amount ranging from about 0.5 to 2%, preferably 1%, by weight of the uncoated tablet. When a colored composition is employed, the preferred application range is 2 to 4%, preferably 3%, by weight of the uncoated tablet. These percentages refer to weight of the dried coating composition.

In accordance with some embodiments of the invention, the coating composition includes additional film-forming materials. Examples of same include hydroxypropyl cellulose, modified starches, modified starch hydrolyzates (such as maltodextrins), gums (such as gellan gum, gum arabic, and so forth) and other materials. In preferred embodiments, when such materials are used, the total amount of film-forming material in the coating composition is in range of 10 to 20%.

Any suitable equipment may be used in conjunction with the invention to coat the tablets. Generally, tablets are coated in a ventilated pan in which the tablets are continuously tumbled. The coating composition is pumped through a spray nozzle, and heated air is passed through the pan to ventilate the pan and to dry the tablets. Any conventional or otherwise suitable equipment may be used in conjunction with the invention.

In some embodiments of the invention, a coating of wax may be applied on top of the tablet coating thus prepared. Preferably, the wax is Carnuba wax, although any suitable wax may be employed in conjunction with these embodiments of the invention. The wax may be applied to enhance sheen and to reduce any tackiness that may be inherent in the coated tablets. In some embodiments of the invention, other coatings or imprints may be applied.

The invention further contemplates a coating composition precursor, the coating composition precursor including hemicelluloses, partially depolymerized hemicelluloses, or mixtures thereof, and a plasticizer, the plasticizer being present in an amount ranging from 10 to 20% by weight of the hemicelluloses or partially depolymerized hemicellulose. This composition is preferably in powdered form, and is suitable for transporting to end users. A method for preparing a coating composition is also contemplated by the invention, the method comprising forming a mixture of hemicelluloses, partially depolymerized hemicelluloses, or mixtures thereof, and a plasticizer. In these embodiments of the invention, the other materials deemed useful for use in conjunction with the invention may be further employed; For instance, the coating composition precursor may be formed with a coloring agent or opacifier.

The following Examples are provided to illustrate the invention, but should not be construed as limiting the scope of the invention.

### EXAMPLE 1

Isolation of Corn Hull Hemicellulose from Corn Hulls

Three hundred pounds of ground corn hulls were added to 400 gallons of water to form a slurry. The pH of the slurry was adjusted to 6.5-7.0 with 50% NaOH. The slurry was jet-cooked continuously at 220-225° F. at 20 PSIG. The resulting cooked slurry was centrifuged in order to separate the washed hulls from the wash water. The washed hulls were added to 400 gallons of water at 180° F. to form a second slurry. The resulting cooked slurry was centrifuged to separate the washed hulls from the wash water.

The washed hulls were added to a reactor containing 420 gallons of 190 proof ethanol and 65 pounds of 50% NaOH. The reactor was sealed and heated to 210-220° F. and held for three hours. The contents of the reactor were then cooled to 70-80° F. and filtered to recover an insoluble holocellulose product.

The holocellulose was added to a reactor containing a solution made by combining 67 gallons of water with 290 gallons of 190 proof ethanol, and the temperature was adjusted to 75-85° F. The pH of the slurry of holocellulose was adjusted to 2.9-3.1 with 1:1 hydrochloric acid, and the slurry was mixed for three hours. The contents of the reactor were then filtered to recover the insoluble holocellulose.

The holocellulose was added to a reactor containing 360 gallons of 190 proof ethanol, and the temperature was adjusted to 75-85° F. The contents of the reactor were then filtered to recover the insoluble holocellulose.

The holocellulose was added to a reactor containing 360 gallons of water. The pH of the slurry of holocellulose was adjusted to 3.4-3.6 with 50% NaOH. The reactor was sealed and heated to 210-220° F. and held for two and one half hours. The contents of the reactor were then filtered to remove the insoluble cellulose from the soluble hemicellulose. The solution of hemicellulose was evaporated to yield a syrup that contained 11.8% solids. The syrup was spray-dried to yield a tan powder.

### EXAMPLE 2

Production of Bleached Treated Hemicellulose from Corn Hulls

Three hundred pounds of ground corn hulls were added to 400 gallons of water to form a slurry. The pH of the slurry was adjusted to 6.5-7.0 with 50% NaOH. The slurry was jet-cooked continuously at 220-225° F. at 20 PSIG. The resulting cooked slurry was centrifuged to separate the washed hulls from the wash water. The washed hulls were added to 400 gallons of water at 180° F. to form a second slurry. The resulting cooked slurry was centrifuged in order to separate the washed hulls from the wash water.

The washed hulls were added to a reactor containing 420 gallons of 190 proof ethanol and 65 pounds of 50% NaOH. The reactor was sealed and heated to 210-220° F. and held for three hours. The contents of the reactor were then cooled to 70-80° F. and filtered to recover an insoluble holocellulose product.

The holocellulose was added to a reactor containing a solution made by combining 67 gallons of water with 290 gallons of 190 proof ethanol, and the temperature was adjusted to 75-85° F. The pH of the slurry of holocellulose was adjusted to 2.9-3.1 with 1:1 hydrochloric acid, and the slurry was mixed for three hours. The contents of the reactor were then filtered to recover the insoluble holocellulose.

The holocellulose was added to a reactor containing 360 gallons of 190 proof ethanol, and the temperature was adjusted to 75-85° F. The contents of the reactor were then filtered to recover the insoluble holocellulose.

The holocellulose was added to a reactor containing 360 gallons of water. The pH of the solution of hemicellulose was adjusted to 10.9-11.1 with 50% NaOH, and 10.6 gallons of 35% hydrogen peroxide were added. The contents of the reactor were heated to 175°-180° F. and held for two hours. The contents of the reactor were then cooled to 70-80° F., and the pH was adjusted to 6.9-7.1 with concentrated hydrochloric acid. The contents of the reactor were then filtered to remove the insoluble cellulose from the soluble hemicellulose. The pH of the solution of hemicellulose was adjusted to 4.4-4.6 with concentrated hydrochloric acid.

The solution of hemicellulose was treated with sodium metabisulfite to neutralize residual oxidant. Seventy two gallons of the solution of hemicellulose containing eighteen pounds of hemicellulose were added to 420 gallons of 190 proof ethanol. The contents of the reactor were then filtered to recover the hemicellulose, which was insoluble in the ethanol:water mixture.

The recovered hemicellulose was dissolved in seventy two gallons of water. The pH of the solution of hemicellulose was adjusted to 4.4-4.6 with concentrated hydrochloric acid, and the solution was added to 420 gallons of 190 proof ethanol. The contents of the reactor were then filtered to recover the hemicellulose.

The recovered hemicellulose was dissolved in seventy two gallons of water. The pH of the solution of hemicellulose was adjusted to 6.9-7.1 with concentrated hydrochloric acid. The solution was spray-dried to give a white powder.

### EXAMPLE 3

Production of Partially Depolymerized Hemicellulose

One hundred sixty two grams dry basis of the corn hull hemicellulose of Example 2 were dissolved into 4,500 ml water at 55° C. The pH was adjusted to 4.80 with 5.8N hydrochloric acid. To the solution was added 3.6 g Genencor Enzyme xylanase AO-3205-GC140, and the mixture was maintained with stirring for 24 hours. A second aliquot of the Genencor enzyme, 3.6 g was added, and the mixture again was maintained with stirring for an additional 24 hours. The enzymes were inactivated by heating the mixture to the boiling temperature.

The system was filtered across a vacuum filter precoated with Celite HYFLO and Celite 577 filter aids. The filtrate was concentrated to a syrup that contained 14.7% solids using a BUCHI Laboratory Evaporator.

### EXAMPLE 4

Semi-Continuous Process for Production of Acid-Hydrolyzed Hemicellulose

Dried U.S. Number 2 grade hybrid yellow dent corn hulls from a corn wet milling process were ground to a particle size suitable for jet cooking. The ground corn hulls (346 pounds, as-is basis), were mixed with 480 gallons of water to form a slurry. To the slurry was added 800 ml NaOH (50%) to achieve a pH of 6.6 at 70° F.

The resulting slurry was continuously jet-cooked in a continuous jet cooker equipped with a Hydroheater Combining Tube which inflicted high shear into the slurry at the point of contact with the high pressure steam at approximately 150 psig. The jet-cooking conditions were as follows:
Temperature: 220-225° F.
Pressure: approx. 20 psig
Retention time: 4.5 minutes.

The cooked corn hulls were recovered from the cooked slurry by passing the cooked slurry across a DSM Screen at high pressure. The filtered cooked corn hulls were added to a well-agitated tank of 360 gallons of water at 180° F.

The cooked corn hulls were recovered a second time from the slurry at 180° F. by passing the slurry at 180° F. across a DSM Screen at high pressure. The DSM filtered cooked corn hulls were added to a well-agitated tank of 360 gallons of water at 180° F. This process was repeated a third time.

Calcium hydroxide (40 pounds) was added to the well agitated slurry. The resulting slurry was continuously jet-cooked in a continuous jet cooker equipped with a Hydroheater Combining Tube which inflicted high shear into the slurry at the point of contact with high pressure steam at approx. 150 psig. The jet-cooking conditions were as follows:
Temperature: 325-335° F.:
Pressure: approx. 95 psig
Retention time: 27 minutes.

The resultant mixture was centrifuged with a Sharples P-660 centrifuge. The hemicellulose solution (or “overs”) was pumped to a continuously stirred tank reactor where 25 lbs of a 35% hydrogen peroxide solution per 140 gallons of hemicellulose solution at 160-170° F. was added. After 90 minutes, the pH of the solution was adjusted to 4.0 with hydrochloric acid, and the solution temperature was adjusted to 120-130° F. and held for 180 minutes. The acid-hydrolyzed, bleached hemicellulose solution was pumped to a continuously stirred tank reactor and cooled to 80-90° F. where sodium metabisulfite was added to neutralize residual oxidant.

Magnesium silicate, “HAZE-OUT”, was added at a rate of 0.75 pounds per 100 gallons of solution, and calcium hydroxide was added to adjust the pH of the mixture to 7.0. The mixture was filtered across a Rotary Vacuum Filter which had been precoated with Celite 503 filter aid. Magnesium silicate was again added to the filtrate at a rate of 0.75 pounds per 100 gallons of solution, and the mixture was filtered across a Niagra Filter Press which had been precoated with Celite 503 filter aid, over polypropylene filter pads having porosity of 1-3μ.

The filtrate was then passed through a 5μ filter, and the temperature was adjusted to 120-130° F. The filtrate was passed through an ultra filtration unit with a 10,000 molecular weight cut-off membrane. The retentate was diafiltered to a conductivity of 700 microSiemens. The ultra filtered retentate was spray dried.

### EXAMPLE 5

Continuous Process for the Production of Acid-Hydrolyzed Hemicellulose

Dried U.S. Number 2 grade hybrid yellow dent corn hulls from a corn wet milling process were ground to a particle size suitable for jet cooking. The ground corn hulls (346 pounds, as-is basis), were mixed with 480 gallons of water to form a slurry. To the slurry was added 800 ml NaOH (50%) to achieve a pH of 6.6 at 70° F.

The resulting slurry was continuously jet-cooked in a continuous jet cooker equipped with a Hydroheater Combining Tube which inflicted high shear into the slurry at the point of contact with the high pressure steam at approximately 150 psig. The jet-cooking conditions were as follows:
Temperature: 220-225° F.
Pressure: approx. 20 psig
Retention time: 4.5 minutes

The cooked corn hulls were recovered from the cooked slurry by passing the cooked slurry across a DSM Screen at high pressure. The filtered cooked corn hulls were added to a well-agitated tank of 360 gallons of water at 180° F. The cooked corn hulls were recovered a second time from the slurry at 180° F. by passing the slurry at 180° F. across a DSM Screen at high pressure. The DSM filtered cooked corn hulls were added to a well-agitated tank of 360 gallons of water at 180° F. This process was repeated a third time.

Calcium hydroxide (40 pounds) was added to the well agitated slurry. The resulting slurry was continuously jet-cooked in a continuous jet cooker equipped with a Hydroheater Combining Tube which inflicted high shear into the slurry at the point of contact with high pressure steam at approx. 150 psig. The jet-cooking conditions were as follows:
Temperature: 325-335° F.:
Pressure: approx. 95 psig
Retention time: 27 minutes.

The resultant cooked paste was jet-cooked a second time with high pressure steam at approx. 150 psig. The jet-cooking conditions were as follows:
Temperature: 325-335° F.:
Pressure: approx. 95 psig
Retention time: 30 seconds.

The solubilized, extractable hemicellulose was separated from the remaining insoluble material by centrifugation with a Sharples P-660 centrifuge. The hemicellulose solution was pumped to a continuously stirred tank reactor where hydrogen peroxide was continuously added. The residence time in the reactor at 180-190° F. was 90 minutes.

The solution of bleached hemicellulose was pumped to a continuously stirred tank reactor, where hydrochloric acid was continuously added to maintain a pH value of 4.0. The residence time in the reactor at 160-170° F. was 90 minutes. The hemicellulose became partially depolymerized upon acid hydrolysis.

The solution of acid-hydrolyzed, bleached hemicellulose was pumped to a continuously stirred tank reactor and cooled to 80-90° F., where sodium metabisulfite was added to neutralize residual oxidant. Magnesium silicate, “HAZE-OUT,” was added at a rate of 0.75 pounds per 100 gallons of solution, and calcium hydroxide was added to adjust the pH of the mixture to 7.0. The mixture was filtered across a Rotary Vacuum Filter which had been precoated with Celite 503 filter aid. Magnesium silicate was added to the filtrate at a rate of 0.75 pounds per 100 gallons of solution, and the mixture was then filtered across a Niagra Filter Press which had been precoated with Celite 503 filter aid over polypropylene filter pads having porosity of 1-3μ. The filtrate was then passed through a 5μ filter, and the temperature was adjusted to 120-130° F. The filtrate was passed through an ultrafiltration unit with a 10,000 molecular weight cut-off membrane. The retentate was diafiltered to a conductivity of 700 microSiemens. The ultrafiltered retentate was spray dried.

### EXAMPLE 6

Tablet Coating Formulation

The products of EXAMPLES 1 and 2 were incorporated into the coating compositions shown in the following table, and these coating compositions were sprayed onto tablets. In this and subsequent compositional tables, the balance of the coating composition was water, and the percentages of the other ingredients are expressed on a weight basis. Examples designated “CA” are comparative examples.

| EXAMPLE | COATING FORMULATION |
|---|---|
| 6-A (CA) | 12.0% INSTANT PURE-COTE B793 |
| | 1.2% Glycerin |
| 6-B | 6.0% Product of EXAMPLE 2 |
| | 6.0% INSTANT PURE-COTE B793 |
| | 1.2% Glycerin |
| 6-C | 6.0% Product of EXAMPLE 1 |
| | 6.0% INSTANT PURE-COTE B793 |
| | 1.2% Glycerin |
| 6-D (CA) | 7.0% Gum Arabic |
| | 7.0% INSTANT PURE-COTE B793 |
| | 1.4% Glycerin |
| | 0.5% Polysorbate 80 |
| 6-E (CA) | 5.0% INSTANT PURE-COTE B793 |
| | 5.0% METHOCEL E-5 Premium HPMC |
| | 0.5% Polysorbate 80 |
| | 1.0% Propylene Glycol |
| | 0.5% B816 Corn Starch |
| | 1.0% Titanium Dioxide |
| | 0.18% Sensient Yellow #6 |
| 6-F | 5.93% Product of EXAMPLE 2 |
| | 5.93% INSTANT PURE-COTE B793 |
| | 1.20% Glycerin |
| | 0.50% Titanium Dioxide |
| | 0.20% Sensient Yellow #6 |
| | 0.50% B815 Corn Starch |
| 6-G (CA) | 7.0% INSTANT PURE-COTE B793 |
| | 7.0% Gum Arabic |
| | 1.4% Glycerin |
| | 0.5% Polysorbate 80 |
| | 0.5% B816 Corn Starch |
| | 1.0% Titanium Dioxide |
| | 0.18% Sensient Yellow #6 |

Tablets were coated in a Hi-Coater Laboratory Development Coating System LDCS 5 manufactured by Vector Corporation, Marion, Iowa. The 1.3 liter, fully perforated, side-vented coating pan was equipped with one gun at a three inch gun to bed distance. The solution spray system comprised one air atomizing spray gun (2850 nozzle/070 aircap) with a peristaltic pump. Air was continuously passed through the tablet bed to provide heating and drying functions.

Tablets used in the coating process were placebos manufactured from an 80/20 lactose/microcrystalline cellulose blend with 0.5% magnesium stearate as the lubricant. They were compressed on a Vector/Colton B2 Rotary Press, Model 2216, equipped with 0.442 inch round tooling with a Vector logo.

A batch core weight of 900 g of uncoated tablets was tumbled in the fully perforated coating pan in order to dedust and heat the tablets to ready them for coating. The coating compositions were sprayed onto the tablets via the solution spray system described above. The coatings were continuously sprayed and dried onto the tablets by the flow of heated air pulled through the tablet bed. The coatings were sprayed onto the tablets at 1.0%, 2.0%, and 3.0% of the weight of the tablets on a dry weight basis. Average operating conditions are shown in the following table.

| | Example | | | | | | |
|---|---|---|---|---|---|---|---|
| | 6-A | 6-B | 6-C | 6-D | 6-E | 6-F | 6-G |
| Inlet Air Temp | 70° C. | 66° C. | 66° C. | 66° C. | 72° C. | 66° C. | 68° C. |
| Exhaust Air Temp | 40° C. | 40° C. | 41° C. | 42° C. | 41° C. | 41° C. | 40° C. |
| Inlet Airflow | 40 cfm | 38 cfm | 38 cfm | 37 cfm | 40 cfm | 38 cfm | 38 cfm |
| Nozzle Pressure | 15 psi | 15 psi | 15 psi | 16 psi | 16 psi | 15 psi | 16 psi |
| Pan Speed | 30 rpm | 25 rpm | 25 rpm | 25 rpm | 25 rpm | 25 rpm | 25 rpm |
| Pump Speed | 10 rpm | 10 rpm | 10 rpm | 10 rpm | 10 rpm | 10 rpm | 10 rpm |

The tablets were tested for disintegration and friability. Disintegration testing was done according to USP physical testing procedure 701 using water maintained at 37+/−2° C. as the immersion fluid and a Van-Kel Industries disintegration tester. Friability testing was performed using a PHARMA TEST friabulator equipped with a Roche wheel. Ten tablets were weighed before and after tumbling for four minutes in the Roche wheel and the percent weight loss was calculated. The following results were obtained.

| EXAMPLE | COATING PERFORMANCE |
|---|---|
| Tablet Core - | DISINTEGRATION = 25 sec, 30 sec |
| NO COATING | FRIABILITY = 0.06 |
| 6-A | At 1% coating tablets looked great. No picking, good coating, edges |
| | OK. At 2% coating tablets look good. Great logo definition and |
| | surface coating; edges OK. Successful trial. |
| | DISINTEGRATION = 50 sec, 55 sec |
| | FRIABILITY = 0.01 |
| 6-B | Sprays very nicely. At 1% coating tablets looked good. At 2% |
| | coating and 3% coating tablets look good. Good adhering coating |
| | but not tacky after drying. Good edges and good surface. Less |
| | gloss than B793, but more than HPMC. Overall success. |
| | DISINTEGRATION = 95 sec, 80 sec |
| | FRIABILITY = 0.02 |
| 6-C | At 1% coating tablets looked very good. At 2% still good coating. |
| | Good adhesion and strong film. At 3% coating very slight edge |
| | wear on a few tablets. Overall good. Good logo definition, good |
| | surface, nice looking tablets. |
| | DISINTEGRATION = 90 sec, 100 sec |
| | FRIABILITY = 0.01 |
| 6-D | At 1% good coating, tacky. 2% tacky, slight edge wear, 3% tacky, |
| | slight edge wear, good surface coating, but edge wear with some |
| | cracks. |
| | DISINTEGRATION = 80 sec, 90 sec |
| | FRIABILITY = 0.11 |
| 6-E | Tablets look good at 2%. At 3% very nice. Good edges, good |
| | surface. Successful run. |
| | DISINTEGRATION = 155 sec, 160 sec |
| | FRIABILITY = 0.00 |
| 6-F | Tablets look good at 2%. Some edge wear. Overall tablets look |
| | good, light gloss, good logo defimtion. |
| | DISINTEGRATION = 85 sec, 75 sec |
| | FRIABILITY = 0.02 |
| 6-G | Solids a little high, slight nozzle trouble. Coating looks good at 2%. |
| | At 3%, the surface looked good, logo definition good, too much |
| | edge wear. Also tablets are slightly tacky. DISINTEGRATION = 80 sec, |
| | 75 sec |
| | FRIABILITY = 0.03 |

### EXAMPLE 7

The product of EXAMPLE 3 was incorporated into the coating compositions shown in the following table.

| EXAMPLE | COATING FORMULATION |
|---|---|
| 7-A (CA) | 10.0% METHOCEL E-5 Premium HPMC |
| | 1.0% Propylene glycol |
| 7-B | 14.46% Product of EXAMPLE 3 |
| | 1.46% Glycerin |
| 7-C | 14.46% Product of EXAMPLE 3 |
| | 1.46% Propylene glycol |
| 7-D (CA) | 10.0% METHOCEL E-5 Premium HPMC |
| | 1.0% Propylene glycol |
| | 0.50% Titanium dioxide |
| | 0.20% Sensient Yellow #6 |
| 7-E | 14.54% Product of EXAMPLE 3 |
| | 1.46% Glycerin |
| | 0.50% Titanium Dioxide |
| | 0.20% Sensient Yellow #6 |
| 7-F | 14.32% Product of EXAMPLE 3 |
| | 1.46% Propylene glycol |
| | 0.20% Polysorbate 80 |
| | 0.50% Titanium Dioxide |
| | 0.20% Sensient Yellow #6 |
| 7-G | 14.35% Product of EXAMPLE 3 |
| | 1.46% Polyethylene Glycol 3350 |
| | 0.50% Titanium Dioxide |
| | 0.20% Sensient Yellow #6 |

Tablets were coated in accordance with the procedure previously described. The coatings were sprayed onto the tablets at 1.0% weight gain for clear coated tablets and 3.0% weight gain for color coated tablets. Average operating conditions are shown in the following table.

| | Example | | | | | | |
|---|---|---|---|---|---|---|---|
| | 7-A | 7-B | 7-C | 7-D | 7-E | 7-F | 7-G |
| Inlet Air Temp | 74° C. | 65° C. | 64° C. | 65° C. | 65° C. | 65° C. | 65° C. |
| Exhaust Air Temp | 42° C. | 40° C. | 41° C. | 46° C. | 41° C. | 43° C. | 42° C. |
| Inlet Airflow | 40 cfm | 40 cfm | 37 cfm | 36 cfm | 38 cfm | 34 cfm | 36 cfm |
| Nozzle Pressure | 15 psi | 15 psi | 15 psi | 15 psi | 15 psi | 15 psi | 15 psi |
| Pan Speed | 18 rpm | 18 rpm | 18 rpm | 15 rpm | 18 rpm | 15 rpm | 15 rpm |
| Pump Speed | 8 rpm | 7 rpm | 7 rpm | 10 rpm | 8 rpm | 8 rpm | 8 rpm |

The tablets were evaluated as described previously. The following results were obtained.

| EXAMPLE | COATING PERFORMANCE |
|---|---|
| Tablet Core - | DISINTEGRATION = 30 seconds |
| NO COATING | FRIABILITY = 0.1% |
| 7-A | Tablets ran very well. Good coating. Good gloss, good edges in general |
| | with very slight edge wear. |
| | DISINTEGRATION = 15 minutes |
| | FRIABILITY = 0% |
| 7-B | Good spray with this higher solids, short time in coating pan; Great |
| | surface coating and gloss, good adhesion, slight edge wear, overall, |
| | excellent coating. |
| | DISINTEGRATION = 17 minutes |
| | FRIABILITY = 0% |
| 7-C | Tablets coated very well; Good gloss, good adhesion, good coating with |
| | very slight edge wear. Overall, good trial. |
| | DISINTEGRATION = 18 minutes |
| | FRIABILITY = 0% |
| 7-D | At 1%, tablets had slight picks and uneven color, but overall a good |
| | coating; At 2%, problems were gone and had a good coating; Final |
| | coating looked great. |
| | DISINTEGRATION = 16 minutes |
| | FRIABILITY = 0% |
| 7-E | At 1%, tablets looked good, with some edge wear; very slight tack; |
| | Final coating has good gloss, good logo definition and good surface |
| | coating with very slight edge wear. |
| | DISINTEGRATION = 17 minutes |
| | FRIABILITY = 0% |
| 7-F | At 1%, tablets have uneven color, At 2% slight picking and slight tack, |
| | Final coating problems were mostly gone, great gloss, slight orange peel |
| | and slight tack, but overall good coating. |
| | DISINTEGRATION = 16 minutes |
| | FRIABILITY = 0% |
| 7-G | At 1%, tablets look good, Final coating looked great, good edges, good |
| | gloss, good adhesion, slight tack, Excellent coating trial. |
| | DISINTEGRATION = 15 minutes |
| | FRIABILITY = 0.02% |

### EXAMPLE 8

The product of EXAMPLE 4 was incorporated into the coating formulations shown in the following table.

| EXAMPLE | COATING FORMULATION |
|---|---|
| 8-A (CA) | 10.0% METHOCEL E-5 Premium HPMC |
| | 1.0% Polyethylene glycol 3350 |
| 8-B | 10.0% Product of EXAMPLE 4 |
| | 1.0% Polyethylene glycol 300 |
| 8-C | 10.0% Product of EXAMPLE 4 |
| | 1.0% Polyethylene glycol 3350 |
| 8-D | 10.0% Product of EXAMPLE 4 |
| | 1.0% Polyethylene glycol 8000 |
| 8-E | 14.0% Product of EXAMPLE 4 |
| | 1.4% Polyethylene glycol 3350 |
| 8-F (CA) | 10.0% METHOCEL E-5 Premium HPMC |
| | 1.0% Polyethylene glycol 3350 |
| | 0.5% Titanium Dioxide |
| | 0.2% Sensient Yellow #6 |
| 8-G | 10.0% Product of EXAMPLE 4 |
| | 1.0% Polyethylene Glycol 300 |
| | 0.5% Titanium Dioxide |
| | 0.2% Sensient Yellow #6 |
| 8-H | 10.0% Product of EXAMPLE 4 |
| | 1.0% Polyethylene glycol 3350 |
| | 0.5% Titanium Dioxide |
| | 0.2% Sensient Yellow #6 |
| 8-I | 14.0% Product of EXAMPLE 4 |
| | 1.4% Polyethylene Glycol 3350 |
| | 0.5% Titanium Dioxide |
| | 0.2% Sensient Yellow #6 |

Tablets were coated in accordance with the procedure previously described. Average conditions were as follows:

| | Example | | | | | | | | | | | | | | | | | |
|---|---|---|---|---|---|---|---|---|---|---|---|---|---|---|---|---|---|---|
| | 8-A | | 8-B | | 8-C | | 8-D | | 8-E | | 8-F | | 8-G | | 8-H | | 8-I | |
| Inlet Air | 75-80° | C. | 75-80° | C. | 74° | C. | 72° | C. | 70° | C. | 78° | C. | 80° | C. | 75° | C. | 73° | C. |
| Temp | | | | | | | | | | | | | | | | | | |
| Exhaust Air | 42° | C. | 44° | C. | 43° | C. | 43° | C. | 42° | C. | 42° | C. | 43-46° | C. | 44° | C. | 43° | C. |
| Temp | | | | | | | | | | | | | | | | | | |
| Inlet | 38 | cfm | 40 | cfm | 40 | cfm | 40 | cfm | 38 | cfm | 43 | cfm | 38 | cfm | 37 | cfm | 32 | cfm |
| Airflow | | | | | | | | | | | | | | | | | | |
| Nozzle | 15 | psi | 15 | psi | 15 | psi | 15 | psi | 15 | psi | 15 | psi | 15 | psi | 15 | psi | 14 | psi |
| Pressure | | | | | | | | | | | | | | | | | | |
| Pan Speed | 20 | rpm | 20 | rpm | 20 | rpm | 20 | rpm | 20 | rpm | 20 | rpm | 20 | rpm | 20 | rpm | 20 | rpm |
| Pump Speed | 8 | rpm | 9 | rpm | 9 | rpm | 9 | rpm | 9 | rpm | 12 | rpm | 10 | rpm | 10 | rpm | 10 | rpm |

The tablets were evaluated as previously described. The following results were obtained.

| EXAMPLE | COATING PERFORMANCE |
|---|---|
| Tablet Core - | DISINTEGRATION = 1 minute |
| NO COATING | FRIABILITY = 0% |
| 8-A | Very good tablets, good gloss, no cracking, good overall coating - |
| | good control. |
| | DISINTEGRATION = 22 minutes |
| | FRIABILITY = 0% |
| 8-B | Tablets look great, good edges, great gloss - better than control, good |
| | logo definition, very slight nozzle build-up; viscosity is lower than |
| | HPMC, slight tackiness. |
| | DISINTEGRATION = 19 minutes |
| | FRIABILITY = 0% |
| 8-C | Very good coating, great gloss, good continuity, good edges, good |
| | logo definition; very slight nozzle build-up and very slight tack. |
| | Overall —great trial. |
| | DISINTEGRATION = 20 minutes |
| | FRIABILITY = 0.2% |
| 8-D | Tablets are tumbling low in the pan due to plasticizer, Tablet coating |
| | looks good, good gloss, good logo definition, good edges, slight |
| | tackiness, slight nozzle build-up. |
| | DISINTEGRATION = 26 minutes |
| | FRIABILITY = 0% |
| 8-E | Good coating, good gloss, good edges, good continuous coating. |
| | Overall great coating trial. |
| | DISINTEGRATION = 48 seconds |
| | FRIABILITY = 0% |
| 8-F | At 1% tablets look good, good final coating - good control. |
| | DISINTEGRATION = 4 minutes |
| | FRIABILITY = 0% |
| 8-G | At 1%, slight smearing on the tablet surface; final tablet coating |
| | looked great, great gloss, good edges, overall very good coating. |
| | DISINTEGRATION = 3 minutes |
| | FRIABILITY = 0% |
| 8-H | At 1%, slight smearing, but it was gone by 2%. Final tablets looked |
| | great, good continuous coating, great gloss, good edges and good logo |
| | definition, very slight tackiness. |
| | DISINTEGRATION = 2 minutes |
| | FRIABILITY = 0% |
| 8-I | At 1% tablets looked good, no smearing, higher viscosity worked well |
| | to spray; Final tablets looked great, great gloss, good continuous |
| | coating, good edges, good logo definition. Overall, successful coating |
| | trial. |
| | DISINTEGRATION = 2 minutes |
| | FRIABILITY = 0% |

### EXAMPLE 9

The product of Example 5 is incorporated into a coating composition that included water, the product of Example 5 (10%) and polyethylene glycol (1%). This composition is used to coat tablets as heretofore described.

### EXAMPLE 10

Examples 6-9 are repeated, except that the tablets are not placebo tablets, but contain 220 mg naproxen sodium.

It is thus seen that hemicellulose, partially depolymerized hemicellulose, and mixtures thereof may be used in tablet coating compositions.

All references cited herein are hereby incorporated by reference in their entireties.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention. No language in the specification should be construed as indicating that any non-claimed element is essential to the practice of the invention.

Preferred embodiments of this invention are described herein, including the best mode known to the inventors for carrying out the invention. Variations of those preferred embodiments may become apparent to those of ordinary skill in the art upon reading the foregoing description. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for coating a tablet, comprising:
providing a tablet, said tablet comprising a material selected from the group consisting of a biologically active material, a biologically inert material, and mixtures thereof;
providing a coating composition; and
coating at least a portion of said tablet with said coating composition, said coating composition comprising a film-forming material selected from the group consisting of hemicellulose, partially depolymerized hemicellulose, and mixtures thereof, said material forming a film over at least said portion of said tablet.

2. A method according to claim 1, said film-forming material being selected from the group consisting of corn hull hemicellulose and partially depolymerized corn hull hemicellulose.

3. A method according to claim 1, said coating composition including water and said film-forming material, said film-forming material being present in said composition in an amount ranging from 10 to 20% by weight.

4. A method according to claim 1, said composition including a plasticizer, said plasticizer being present in an amount of about 10 to 20% by weight of said film-forming material.

5. A method according to claim 4, said plasticizer being selected from the group consisting of glycerin, polyethylene glycol, and propylene glycol.

6. A method according to claim 1, said material including at least one other film-forming material, said other film-forming material being selected from the group consisting of starch, natural gums, and modified celluloses.

7. A method according to claim 1, said coating composition including at least one coloring agent.

8. A method according to claim 1, said coating composition including an opacifier.

9. A method according to claim 1, said coating composition including a detackifier.

10. A method according to claim 1, said film-forming material comprising hemicellulose.

11. A method according to claim 1, said film-forming material comprising partially depolymerized hemicellulose.

12. A method according to claim 1, said coating having a weight ranging from 0.5 to 5% by weight of said tablet absent said coating.

13. A coated tablet comprising:
a tablet comprising a material selected from the group consisting of a biologically active material, a biologically inert material, and mixtures thereof; and
a coating disposed over at least a portion of said tablet, said coating comprising a film formed from a film-forming material, said film-forming material selected from the group consisting of hemicellulose, partially depolymerized hemicellulose, and mixtures thereof.

14. A coated tablet according to claim 13, said film-forming material being selected from the group consisting of corn hull hemicellulose and partially depolymerized corn hull hemicellulose.

15. A coated tablet according to claim 13, said coating further including at least one other film-forming material, said other film-forming material being selected from the group consisting of starch, natural gums, and modified celluloses.

16. A coated tablet according to claim 13, said coating including at least one coloring agent.

17. A coated tablet according to claim 13, said coating including an opacifier.

18. A coated tablet according to claim 13, said coating including a detackifier.

19. A coated tablet according to claim 13, said film-forming material comprising hemicellulose.

20. A coated tablet according to claim 13, said film-forming material comprising partially depolymerized hemicellulose.

21. A coated tablet according to claim 13, said coating having a weight ranging from 0.5 to 5% by weight of said tablet absent said coating.

22. A method for preparing a coating composition, comprising forming a mixture of water, a plasticizer, and a film-forming material selected from the group consisting of hemicellulose, partially depolymerized hemicellulose, and mixtures thereof, said film-forming material being present in said mixture in an amount ranging from 10 to 20%, said plasticizer being present in an amount of about 10 to 20% by weight of said film-forming material.

23. A coating composition prepared in accordance with claim 24.

24. A coating composition comprising a film-forming material selected from the group consisting of hemicellulose, partially depolymerized hemicellulose, and mixtures thereof; and a plasticizer, said material being at least substantially dry and being in powdered form.
